SEQUENCE LISTING (12 sequences)

SEQ ID NO: 1 - protein, 494 aa, Homo sapiens
MLTSKGQGFLHGGLCLWLCVFTPFFKGCVGCATEERLFHKLFSHYNQFIRPVENVSDPVTVHFEVAITQLANVDEVNQIMETNLWLRHIWNDYKLRWDPMEYDGIETLRVPADKIWKPDIVLYNNAVGDFQVEGKTKALLKYNGMITWTPPAIFKSSCPMDITFFPFDHQNCSLKFGSWTYDKAEIDLLIIGSKVDMNDFWENSEWEIIDASGYKHDIKYNCCEEIYTDITYSFYIRRLPMFYTINLIIPCLFISFLTVLVFYLPSDCGEKVTLCISVLLSLTVFLLVITETIPSTSLVVPLVGEYLLFTMIFVTLSIVVTVFVLNIHYRTPTTHTMPRWVKTVFLKLLPQVLLMRWPLDKTRGTGSDAVPRGLARRPAKGKLASHGEPRHLKECFHCHKSNELATSKRRLSHQPLQWVVENSEHSPEVEDVINSVQFIAENMKSHNETKEVEDDWKYVAMVVDRVFLWVFIIVCVFGTAGLFLQPLLGNTGKS

SEQ ID NO: 2 - protein, 128 aa, Homo sapiens
PRWVKTVFLKLLPQVLLMRWPLDKTRGTGSDAVPRGLARRPAKGKLASHGEPRHLKECFHCHKSNELATSKRRLSHQPLQWVVENSEHSPEVEDVINSVQFIAENMKSHNETKEVEDDWKYVAMVVDR

SEQ ID NO: 3 - protein, 505 aa, Homo sapiens
MGSGPLSLPLALSPPRLLLLLLLSLLPVARASEAEHRLFERLFEDYNEIIRPVANVSDPVIIHFEVSMSQLVKVDEVNQIMETNLWLKQIWNDYKLKWNPSDYGGAEFMRVPAQKIWKPDIVLYNNAVGDFQVDDKTKALLKYTGEVTWIPPAIFKSSCKIDVTYFPFDYQNCTMKFGSWSYDKAKIDLVLIGSSMNLKDYWESGEWAIIKAPGYKHDIKYNCCEEIYPDITYSLYIRRLPLFYTINLIIPCLLISFLTVLVFYLPSDCGEKVTLCISVLLSLTVFLLVITETIPSTSLVIPLIGEYLLFTMIFVTLSIVITVFVLNVHYRTPTTHTMPSWVKTVFLNLLPRVMFMTRPTSNEGNAQKPRPLYGAELSNLNCFSRAESKGCKEGYPCQDGMCGYCHHRRIKISNFSANLTRSSSSESVDAVLSLSALSPEIKEAIQSVKYIAENMKAQNEAKEIQDDWKYVAMVIDRIFLWVFTLVCILGTAGLFLQPLMAREDA

SEQ ID NO: 4 - protein, 136 aa, Homo sapiens
HYRTPTTHTMPSWVKTVFLNLLPRVMFMTRPTSNEGNAQKPRPLYGAELSNLNCFSRAESKGCKEGYPCQDGMCGYCHHRRIKISNFSANLTRSSSSESVDAVLSLSALSPEIKEAIQSVKYIAENMKAQNEAKEI

SEQ ID NO: 5 - protein, 502 aa, synthetic construct
MLTSKGQGFLHGGLCLWLCVFTPFFKGCVGCATEERLFHKLFSHYNQFIRPVENVSDPVTVHFEVAITQLANVDEVNQIMETNLWLRHIWNDYKLRWDPMEYDGIETLRVPADKIWKPDIVLYNNAVGDFQVEGKTKALLKYNGMITWTPPAIFKSSCPMDITFFPFDHQNCSLKFGSWTYDKAEIDLLIIGSKVDMNDFWENSEWEIIDASGYKHDIKYNCCEEIYTDITYSFYIRRLPMFYTINLIIPCLFISFLTVLVFYLPSDCGEKVTLCISVLLSLTVFLLVITETIPSTSLVVPLVGEYLLFTMIFVTLSIVVTVFVLNIHYRTPTTHTMHYRTPTTHTMPSWVKTVFLNLLPRVMFMTRPTSNEGNAQKPRPLYGAELSNLNCFSRAESKGCKEGYPCQDGMCGYCHHRRIKISNFSANLTRSSSSESVDAVLSLSALSPEIKEAIQSVKYIAENMKAQNEAKEIVFLWVFIIVCVFGTAGLFLQPLLGNTGKS

SEQ ID NO: 6 - protein, 502 aa, Homo sapiens
MARRCGPVALLLGFGLLRLCSGVWGTDTEERLVEHLLDPSRYNKLIRPATNGSELVTVQLMVSLAQLISVHEREQIMTTNVWLTQEWEDYRLTWKPEEFDNMKKVRLPSKHIWLPDVVLYNNADGMYEVSFYSNAVVSYDGSIFWLPPAIYKSACKIEVKHFPFDQQNCTMKFRSWTYDRTEIDLVLKSEVASLDDFTPSGEWDIVALPGRRNENPDDSTYVDITYDFIIRRKPLFYTINLIIPCVLITSLAILVFYLPSDCGEKMTLCISVLLALTVFLLLISKIVPPTSLDVPLVGKYLMFTMVLVTFSIVTSVCVLNVHHRSPTTHTMAPWVKVVFLEKLPALLFMQQPRHHCARQRLRLRRRQREREGAGALFFREAPGADSCTCFVNRASVQGLAGAFGAEPAPVAGPGRSGEPCGCGLREAVDGVRFIADHMRSEDDDQSVSEDWKYVAMVIDRLFLWIFVFVCVFGTIGMFLQPLFQNYTTTTFLHSDHSAPSSK

SEQ ID NO: 7 - protein, 458 aa, Homo sapiens
MLPDFMLVLIVLGIPSSATTGFNSIAENEDALLRHLFQGYQKWVRPVLHSNDTIKVYFGLKISQLVDVDEKNQLMTTNVWLKQEWTDHKLRWNPDDYGGIHSIKVPSESLWLPDIVLFENADGRFEGSLMTKVIVKSNGTVVWTPPASYKSSCTMDVTFFPFDRQNCSMKFGSWTYDGTMVDLILINENVDRKDFFDNGEWEILNAKGMKGNRRDGVYSYPFITYSFVLRRLPLFYTLFLIIPCLGLSFLTVLVFYLPSDEGEKLSLSTSVLVSLTVFLLVIEEIIPSSSKVIPLIGEYLLFIMIFVTLSIIVTVFVINVHHRSSSTYHPMAPWVKRLFLQKLPKLLCMKDHVDRYSSPEKEESQPVVKGKVLEKKKQKQLSDGEKVLVAFLEKAADSIRYISRHVKKEHFISQVVQDWKFVAQVLDRIFLWLFLIVSVTGSVLIFTPALKMWLHSYH

SEQ ID NO: 8 - protein, 827 aa, Homo sapiens
MGSAQLCAGVCDRCHTGEGAGEAWRVLHSARGDLRGEAGGAPVSRDPGLCSLKGNCGRGALAVPPQNPHGPSNVRGESVPPRTPPPPAHAAHLGLQSREFQDTPSCVPGTSGPGEGPPPQFRMQPTATMATAATTTTTTTATVALTTSWDNATGRPTAEPDPILDNYVLLVVVMSLFVGGTLVVLSGVLLLCKRCWDVHQRLNRAMEEAEKTTTTYLDNGTHPAQDPDFRGEDPECQDAETERFLSTSSTGRRVSFNEAALFEQSRKTQDKGRRYTLTEGDFHHLKNARLTHLHLPPLKIVTIHECDSGEASSATTPHPATSPKATLAIFQPPGKALTGRSVGPSSALPGDPYNSAAGATDFAEISPSASSDSGEGTSLDAGTRSTKAGGPGAAAGPGEAGPGSGAGTVLQFLTRLRRHASLDGASPYFKVKKWKLEPSQRAASLDTRGSPKRHHFQRQRAASESTEQEEGDAPQEDFIQYIARAGDAVAFPHPRPFLASPPPALGRLEAAEAAGGASPDSPPERGAGSAGPEQQQPPLEPDAERDAGPEQAQTSYRDLWSLRASLELHAAASDHSSSGNDRDSVRSGDSSGSGSGGAAPAFPPPSPPAPRPKDGEARRLLQMDSGYASIEGRGAGDDTEPPAAPARPRSPRAWPRRPRRDYSIDEKTDALFHEFLRHDPHFDDTPAAARHRARAHPHARKQWQRGRQHSDPGARAAPALAGTPAPPAGAARPARAPLRRGDSVDGPPDGRTLGGAGDDPAIPVIEEEPGGGGCPGSGLCVLPSGSVLDKLAAGLDERLFPPRLAEPVVATPALVAAAPTSPDHSPA

SEQ ID NO: 9 - protein, 365 aa, Homo sapiens
MDGPAEPQIPGLWDTYEDDISEISQKLPGEYFRYKGVPFPVGLYSLESISLAENTQDVRDDDIFIITYPKSGTTWMIEIICLILKEGDPSWIRSVPIWERAPWCETIVGAFSLPDQYSPRLMSSHLPIQIFTKAFFSSKAKVIYMGRNPRDVVVSLYHYSKIAGQLKDPGTPDQFLRDFLKGEVQFGSWFDHIKGWLRMKGKDNFLFITYEELQQDLQGSVERICGFLGRPLGKEALGSVVAHSTFSAMKANTMSNYTLLPPSLLDHRRGAFLRKGVCGDWKNHFTVAQSEAFDRAYRKQMRGMPTFPWDEDPEEDGSPDPEPSPEPEPKPSLEPNTSLEREPRPNSSPSPSPGQASETPHPRPS

SEQ ID NO: 10 - protein, 280 aa, Homo sapiens
MDLQGRGVPSIDRLRVLLMLFHTMAQIMAEQEVENLSGLSTNPEKDIFVVRENGTTCLMAEFAAKFIVPYDVWASNYVDLITEQADIALTRGAEVKGRCGHSQSELQVFWVDRAYALKMLFVKESHNMSKGPEATWRLSKVQFVYDSSEKTHFKDAVSAGKHTANSHHLSALVTPAGKSYECQAQQTISLASSDPQKTVTMILSAVHIQPFDIISDFVFSEEHKCPVDEREQLEETLPLILGLILGLVIMVTLAIYHVHHKMTANQVQIPRDRSQYKHMG

SEQ ID NO: 11 - protein, 630 aa, Homo sapiens
MAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHVTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 12 - protein, 167 aa, Homo sapiens
MASPRTVTIVALSVALGLFFVFMGTIKLTPRLSKDAYSEMKRAYKSYVRALPLLKKMGINSILLRKSIGALEVACGIVMTLVPGRPKDVANFFLLLLVLAVLFFHQLVGDPLKRYAHALVFGILLTCRLLIARKPEDRSSEKKPLPGNAEEQPSLYEKAPQGKVKVS

(19) United States
(12) Patent Application Publication
(10) Pub. No.: US 2025/0189514 A1
(43) Pub. Date: Jun. 12, 2025

(54) EXPRESSION SYSTEMS FOR THE ALPHA6-CONTAINING NICOTINIC ACETYLCHOLINE RECEPTOR AND METHODS OF USE THEREOF

(71) Applicant: Janssen Pharmaceutica NV, Beerse (BE)

(72) Inventors: David Bredt, La Jolla, CA (US); Jose Matta, San Diego, CA (US)

(21) Appl. No.: 18/837,469

(22) PCT Filed: Feb. 10, 2023
(86) PCT No.: PCT/EP2023/053378

§ 371 Date: Aug. 9, 2024

Related U.S. Application Data
(60) Provisional application No. 63/309,092, filed on Feb. 11, 2022.

(51) Int. Cl. G01N 33/50; C07K 14/47; C07K 14/705; C12N 5/071; C12N 9/10

(52) U.S. Cl. CPC G01N 33/5035; C07K 14/4703; C07K 14/70571; C12N 5/0686; C12N 9/1029; C12N 9/13; G01N 33/5041; C12N 2503/02; C12N 2510/00; C12Y 203/01006; C12Y 208/02002; G01N 2333/70571

(57) ABSTRACT

Disclosed herein are isolated recombinant cells for the expression of α6 containing nicotinic acetylcholine receptors (nAChR) and methods of use thereof.

### CROSS REFERENCE TO RELATED APPLICATION

This application is a National Stage of International Application No. PCT/EP2023/053378 filed on Feb. 10, 2023, which claims the benefit of priority to U.S. Provisional Application No. 63/309,092, filed on Feb. 11, 2022, each of which is hereby incorporated by reference in their entireties for all purposes.

### FIELD OF INVENTION

This invention relates to isolated recombinant cells for the expression of α6-containing nicotinic acetylcholine receptor (nAChR) and methods of use thereof.

### REFERENCE TO SEQUENCE LISTING SUBMITTED ELECTRONICALLY

The instant application contains a Sequence Listing which has been submitted electronically in ST.26 format and is hereby incorporated by reference in its entirety. Said ST.26 copy, created on 26 Jan. 2023, is named JAB7137WOPCT1_SL.XML and is 16,108 bytes in size.

### BACKGROUND OF INVENTION

Major depression is characterized by debilitating symptoms that include hopelessness and anhedonia. There is strong evidence linking the dopamine system from the ventral tegmental area to the nucleus accumbens (mesolimbic dopamine system) with reward-related, hedonic, and motivated behaviors. Currently, therapeutic interventions to specifically target dopamine neurons are not available.

Acetylcholine (ACh) plays a major role in the modulation of the dopamine neurons. ACh binds to two major receptors; ionotropic nicotinic (nAChRs) and metabotropic muscarinic (mAChRs) acetylcholine receptors. nAChRs are non-selective cation channels that depolarize dopamine neurons and flux calcium into the cell. This depolarization leads to the generation of action potentials at high frequencies, termed burst firing (>20 Hz). It is this burst firing of the dopamine neurons which codes the physiological roles of the mesolimbic dopamine system with regards to the behaviors mentioned above. Thus modulating nAChRs may lead to therapeutic intervention for mood disorders.

nAChRs are membrane-bound complexes assembled from five subunits, each consisting of a large extracellular N-terminal domain (NTD), a transmembrane domain (TMD) consisting of four transmembrane α-helices (TM1-TM4) connected by intracellular and extracellular loops, including a large second intracellular loop (ICL), and a short extracellular C terminus. Thus, the pentameric nAChR complex comprises three structural entities: an extracellular domain containing the orthosteric sites, a transmembrane domain containing the ion channel, and an intracellular domain, the three entities being assembled from the NTDs, the TMDs, and the ICLs of the five subunits, respectively.

nAChRs containing nAChR subunit α6 (CHRNA6, accession number: NM_004198) are selectively expressed in dopamine neurons and offer a therapeutic target for the modulation of the mesolimbic dopamine system to treat mood disorders. However, drug discovery efforts have been hampered by the inability to express α6-containing nAChRs in recombinant systems typically used for such screens.

There still is a need to develop a recombinant cell line that expresses α6-containing nAChRs robustly and can be used for drug discovery.

### BRIEF SUMMARY OF THE INVENTION

Provided herein are isolated recombinant cells comprising: a) a heterologous nucleic acid encoding an α6 subunit of nAChR; b) a heterologous nucleic acid encoding BARP; c) a heterologous nucleic acid encoding SULT2B1; d) a heterologous nucleic acid encoding LAMP5; e) a heterologous nucleic acid encoding CHAT; and f) a heterologous nucleic acid encoding NACHO.

In one embodiment of the isolated recombinant cell, it further comprises: g) a heterologous nucleic acid encoding a 32 subunit of nAChR; and h) a heterologous nucleic acid encoding a 3 subunit of nAChR, wherein, the α6, 32, and 33 subunits of nAChR form an α6B2B3 nAChR.

In a further embodiment of the isolated recombinant cell, the 6 subunit of nAChR is an 6/3 chimera in which a full or partial sequence of a second intracellular loop (ICL) of the α6 subunit is replaced by a corresponding sequence of a second ICL of an α3 subunit of nAChR, and wherein the α6/3 chimera and the β2 and B3 subunits of nAChR form a chimeric α6/3B233 nAChR.

In a yet further embodiment of the isolated recombinant cell, the recombinant cell is a mammalian cell.

In a yet further embodiment of the isolated recombinant cell, the mammalian cell is selected from the group consisting of a human embryonic kidney 293T (HEK293T) cell, a HEK293F cell, a HeLa cell, a Chinese hamster ovary (CHO) cell, a NIH 3T3 cell, a MCF-7 cell, a Hep G2 cell, a baby hamster kidney (BHK) cell, and a Cos7 cell.

In a yet further embodiment of the isolated recombinant cell, the α6 subunit of nAChR comprises an amino acid sequence with at least 95% identity to the amino acid sequence of SEQ ID NO: 1.

In a yet further embodiment of the isolated recombinant cell, the β2 subunit of nAChR comprises an amino acid sequence with at least 95% identity to the amino acid sequence of SEQ ID NO: 6.

In a yet further embodiment of the isolated recombinant cell, the β3 subunit of nAChR comprises an amino acid sequence with at least 95% identity to the amino acid sequence of SEQ ID NO: 7.

In a yet further embodiment of the isolated recombinant cell, the second ICL of the α6 subunit comprises an amino acid sequence with at least 95% identity to the amino acid sequence of SEQ ID NO: 2 and the second ICL of the α3 subunit comprises an amino acid sequence with at least 95% identity to the amino acid sequence of SEQ ID NO: 4.

In a yet further embodiment of the isolated recombinant cell, the α6/3 chimera comprises an amino acid sequence with at least 95% identity to the amino acid sequence of SEQ ID NO: 5.

In a yet further embodiment of the isolated recombinant cell, the BARP comprises an amino acid sequence with at least 95% identity to the amino acid sequence of SEQ ID NO: 8.

In a yet further embodiment of the isolated recombinant cell, the SULT2B1 comprises an amino acid sequence with at least 95% identity to the amino acid sequence of SEQ ID NO: 9.

In a yet further embodiment of the isolated recombinant cell, the LAMP5 comprises an amino acid sequence with at least 95% identity to the amino acid sequence of SEQ ID NO: 10.

In a yet further embodiment of the isolated recombinant cell, the CHAT comprises an amino acid sequence with at least 95% identity to the amino acid sequence of SEQ ID NO: 11.

In a yet further embodiment of the isolated recombinant cell, the NACHO comprises an amino acid sequence with at least 95% identity to the amino acid sequence of SEQ ID NO: 12.

Further provided herein is a method for identifying agonists, antagonists, or positive allosteric modulators of α6 containing nAChR, the method comprising: a) contacting the isolated recombinant cell as provided above with an agent; and b) determining the activity of the α6 containing nAChR of the isolated recombinant cell, wherein the agent is identified as an agonist or positive allosteric modulator (PAM) if the agent enhances the activity of the α6 containing nAChR and the agent is identified as an antagonist if the agent decreases the activity of the α6 containing nAChR as compared to the activity of the α6 containing nAChR when the isolated recombinant cell was not contacted with the agent.

Yet further provided herein is a method for identifying agonists, antagonists, or positive allosteric modulators of α6β2β3 nAChR, the method comprising: a) contacting the isolated recombinant cell as provided above with an agent; and b) determining the activity of the α6B2B3 nAChR of the isolated recombinant cell, wherein the agent is identified as an agonist or positive allosteric modulator (PAM) if the agent enhances the activity of the 6B2B3 nAChR and the agent is identified as an antagonist if the agent decreases the activity of the α6β2β3 nAChR as compared to the activity of the α6B233 nAChR when the isolated recombinant cell was not contacted with the agent.

In one embodiment of the above method, step b) comprises determining calcium flux of the isolated recombinant cell, wherein the agent is identified as an agonist if the agent enhances the calcium flux as compared to the calcium flux when the isolated recombinant cell was not contacted with the agent.

In a further embodiment of the above method, step b) comprises determining calcium flux and nicotine-evoked calcium flux of the isolated recombinant cell, wherein the agent is identified as an PAM if the agent does not enhance calcium flux and enhances the nicotine-evoked calcium flux as compared to the calcium flux and nicotine-evoked calcium flux when the isolated recombinant cell was not contacted with the agent.

In a yet further embodiment of the above method, step b) comprises determining nicotine-evoked calcium flux of the isolated recombinant cell, wherein the agent is identified as an antagonist if the agent decreases the nicotine-evoked calcium flux as compared to the nicotine-evoked calcium flux when the isolated recombinant cell was not contacted with the agent.

In a yet further embodiment of the method provided above, the isolated recombinant cell is incubated at about 25° C.-35° C. for about 20-50 hours prior to being contacted with the agent.

In a yet further embodiment of the method provided above, the agent is a small molecule or peptide.

Yet further provided herein is a kit comprising (i) the isolated recombinant cell as provided above, and (ii) instructions for use.

### BRIEF DESCRIPTION OF THE DRAWINGS

FIG. 1A is a graph showing functional expression of α6 β2β3 nAChR when co-expressed with different combinations of chaperon proteins (BARP+SULT2B1+LAMP5+NACHO or BARP+SULT2B1+LAMP5+NACHO+CHAT) and incubated at 30° C. prior to testing.

FIG. 1B is a graph showing functional expression of α6/3B2B3 nAChR when co-expressed with different combinations of chaperon proteins (BARP+SULT2B1+LAMP5+NACHO or BARP+SULT2B1+LAMP5+NACHO+CHAT) and incubated at 37° C. prior to testing.

FIG. 1C is a graph showing functional expression of α6/3B2B3 nAChR when co-expressed with different combinations of chaperon proteins (BARP+SULT2B1+LAMP5+NACHO or BARP+SULT2B1+LAMP5+NACHO+CHAT) and incubated at 30° C. prior to testing.

FIG. 2A is a plot of FLIPR signals of the recombinant cells expressing α6/3B2B3 after first incubation with DHbE (a known antagonist of α6-containing nAChR) at various concentrations and second incubation with nicotine.

FIG. 2B is a plot of FLIPR signals of the recombinant cells expressing α6/3B2B3 after first incubation with MLA (a known antagonist of α6-containing nAChR) at various concentrations and second incubation with nicotine.

FIG. 2C is a plot of FLIPR signals of the recombinant cells expressing α6/3B233 after first incubation with mecamylamine (a known antagonist of α6-containing nAChR) at various concentrations and second incubation with nicotine.

FIG. 2D is a plot of FLIPR signals of the recombinant cells expressing α6/3B2B3 after first incubation with aconotoxin MII (a known antagonist of α6-containing nAChR) at various concentrations and second incubation with nicotine.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/ml includes 0.9 mg/ml to 1.1 mg/ml. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of’ or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2 111.03.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F.M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1992)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Isolated Recombinant Cells and Methods of Making Recombinant Cells

The inventions disclosed herein are based, at least in part, on the unexpected finding that co-expressing certain chaperone proteins with α6-containing nicotinic acetylcholine receptors (nAChR) (such as α6ß23 nAChR) in cells generated cells that highly express α6-containing nAChR, making the cells useful for drug discovery. Provided herein are methods of making recombinant cells expressing α6 subunit of nAChR and isolated recombinant cells for the expression of α6 subunit of nAChR. In some embodiments, the α6 subunit may have its second ICL partially or fully replaced by the corresponding amino acid sequence of the second ICL of an α3 subunit of nAChR, which may be termed an α6/3 chimera protein. Also provided herein are methods of making recombinant cells expressing α6B2B3 nAChR and isolated recombinant cells for the expression of α6β2β3 nAChR. In some embodiments, the α6 subunit may be the α6/3 chimera protein as described above, and the recombinant cells expresses α6/3B2B3 nAChR.

As used herein, the terms “α6B2B3 nicotinic acetylcholine receptor”, “α6B2B3 nAChR”, “alpha6beta2beta3 nicotinic acetylcholine receptor”, and “alpha6beta2beta3 nAChR” are used interchangeably and refer to the α6B2B3 nicotinic acetylcholine receptor protein, preferably the human α6B2B3 nAChR, which is a member of a protein family of cholinergic receptors.

α6B2B3 nAChR is a ligand-gated ion channel composed of α6, 32, and B3 subunits. The α6 subunit is encoded by the gene CHRNA6 (NM_004198), the β2 subunit is encoded by the gene CHRNB2 (NM_000748), and the 33 subunit is encoded by the gene CHRNB3 (NM_000749). When expressed together, the subunits co-assemble to form an α6β2β3 nAChR.

In accordance with the present invention, the term α6β2β3 nAChR also encompasses chimeric α6/3323 nAChR, in which the second ICL of the α6 subunit is partially or fully replaced by the corresponding amino acid sequence of the second ICL of the α3 subunit of nAChR. In one embodiment, a portion of the 2^{nd} ICL of the α6 subunit is replaced by the amino acid sequence of the second ICL of the α3 subunit of nAChR. The α3 subunit is encoded by the gene CHRNA3 (NM_000743)

“Recombinant cells” refers to one or more individual cells as well as to a recombinant cell line in which the cells are heterologously expressing protein(s). As used herein, “heterologous expression” of a protein in a cell refers to modifying the cell to express the protein by introducing an exogenous nucleic acid into the cell, e.g., an exogenous nucleic acid that encodes the protein to be expressed. A “heterologous nucleic acid” refers to a nucleic acid exogenous to a cell that is introduced into the cell. In some embodiments, the heterologous nucleic acid is DNA. In some embodiments, the heterologous nucleic acid is RNA. Heterologous expression of a protein in a cell can be achieved using a variety of methods. For example, an expression vector comprising a nucleic acid encoding the protein that is operably linked to a nucleic acid encoding a promoter capable of driving expression of the protein (e.g., a constitutive promoter) may be introduced into the cell.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

In a general aspect, the invention relates to methods of making or generating cells expressing α6 containing nAChR (such as an α6B2B3 nAChR, including chimeric α6/3B2B3 nAChR as described above) that are useful for drug discovery. In one embodiment, the method comprises introducing into a cell a nucleic acid encoding α6 subunit of nAChR (including a chimeric α6/3 subunit), a nucleic acid encoding β-anchoring and -regulatory protein (BARP; accession number: NM_152769), a nucleic acid encoding Sulfotransferase Family 2B Member 1 (SULT2B1; accession number: NM_177973), a nucleic acid encoding lysosomal-associated membrane protein 5 (LAMP5; accession number: NM_012261), a nucleic acid encoding Choline O-Acetyltransferase (CHAT; accession number: NM_020984), and a nucleic acid encoding transmembrane protein 35 (TMEM35; also known as NACHO; accession number: NM_021637). The nucleic acids encoding the α6 subunit of nAChR (including a chimeric α6/3 subunit) and the chaperone proteins (i.e., BARP, SULT2B1, LAMP5, CHAT, and NACHO) can be in an expression vector (e.g., in a single expression vector or in separate expression vectors). In some embodiments, the nucleic acid encoding the α6 subunit of nAChR (including a chimeric α6/3 subunit) and the chaperone proteins (i.e., BARP, SULT2B1, LAMP5, CHAT, and NACHO) is operably linked to a promoter capable of driving expression of the respective protein. In some embodiments, the promoter is a constitutive promoter.

In another aspect, the method comprises introducing into a cell a nucleic acid encoding α6 subunit of α6B2B3 nAChR (including a chimeric α6/3 subunit as described above), a nucleic acid encoding 32 subunit of α6B2B3 nAChR, a nucleic acid encoding 33 subunit of α6B2B3 nAChR, a nucleic acid encoding BARP, a nucleic acid encoding SULT2B1, a nucleic acid encoding LAMP5, a nucleic acid encoding CHAT, and a nucleic acid encoding NACHO, wherein the cell generated by the method expresses α6B2B3 nAChR (including a chimeric α6/3 nAChR as described above) at an increased level compared to the same cell without the nucleic acids encoding BARP, SULT2B1, LAMP5, CHAT, and NACHO.

In another aspect, the invention relates to cells genetically modified to express α6 (including chimeric α6/3 as described above), 2, and 33 subunits of nAChR, BARP, SULT2B1, LAMP5, CHAT, and NACHO, wherein the genetically modified cell expresses these proteins at an increased level relative to the expression of the same protein in the unmodified cell under the same (or substantially the same) conditions.

In another aspect, the invention relates to isolated recombinant cells comprising at least one expression vector selected from the group consisting of an expression vector comprising a nucleic acid sequence encoding BARP, an expression vector comprising a nucleic acid sequence encoding SULT2B1, an expression vector comprising a nucleic acid sequence encoding LAMP5, an expression vector comprising a nucleic acid sequence encoding CHAT, and an expression vector comprising a nucleic acid sequence encoding NACHO.

In a further aspect, the invention relates to isolated recombinant cells comprising a heterologous nucleic acid encoding an α6 subunit of nAChR (including a chimeric α6/3 subunit of nAChR as described above), a heterologous nucleic acid encoding an β2 subunit of nAChR, a heterologous nucleic acid encoding β3 subunit of nAChR, a heterologous nucleic acid encoding BARP, a heterologous nucleic acid encoding SULT2B1, a heterologous nucleic acid encoding LAMP5, a heterologous nucleic acid encoding CHAT, and a heterologous nucleic acid encoding NACHO, wherein the α6 (including α6/3), β2, and β3 subunits forms an α6β2β3 nAChR (or chimeric α6/3β2β3 nAChR). In one embodiment, the heterologous nucleic acids are introduced into the recombinant cells in the form of expression vectors. In one embodiment, the isolated recombinant cells disclosed herein comprises an expression vector comprising the nucleic acid encoding the α6 subunit of nAChR (including the nucleic acid encoding the α6/3 subunit as described above), an expression vector comprising the nucleic acid encoding the β2 subunit of nAChR, an expression vector comprising the nucleic acid encoding the β3 subunit of nAChR, an expression vector comprising the nucleic acid encoding BARP, an expression vector comprising the nucleic acid encoding SULT2β1, an expression vector comprising the nucleic acid encoding LAMP5, an expression vector comprising the nucleic acid encoding CHAT, and an expression vector comprising the nucleic acid encoding NACHO.

In any one of the embodiments described herein, the nucleic acid(s) encoding any one or more of the α6 (including α6/3), 32, and β3 subunits of nAChR, BARP, SULT2β1, LAMP5, CHAT, and NACHO can be in a single expression vector or in separate expression vectors.

In one embodiment, the α6 subunit of nAChR comprises an amino acid sequence with at least 85%, or at least 90%, or at least 95%, such as 95%, 96%, 97%, 98%, 99%, or 100% identity to the amino acid sequence of SEQ ID NO: 1 (NM_004198.2/NP_004189).

| | |
|---|---|
| 1 | mltskgqgfl hgglclwlcv ftpffkgcvg cateerlfhk |
| | lfshynqfir pvenvsdpvt |
| 61 | vhfevaitql anvdevnqim etnlwlrhiw ndyklrwdpm |
| | eydgietlrv padkiwkpdi |
| 121 | vlynnavgdf qvegktkall kyngmitwtp paifksscpm |
| | ditffpfdhq ncslkfgswt |
| 181 | ydkaeidlli igskvdmndf wenseweiid asgykhdiky |
| | ncceeiytdi tysfyirrlp |
| 241 | mfytinliip clfisfltvl vfylpsdcge kvtlcisvll |
| | sltvfllvit etipstslvv |
| 301 | plvgeyllft mifvtlsivv tvivlnihyr tptthtmprw |
| | vktvflkllp qvllmrwpld |
| 361 | ktrgtgsdav prglarrpak gklashgepr hlkecfhchk |
| | snelatskrr lshqplqwvv |
| 421 | ensehspeve dvinsvqfia enmkshnetk eveddwkyva |
| | mvvdrvflwv fiivcvfgta |
| 481 | glflqpllgn tgks |

In one embodiment, the 2^{nd} ICL of the α6 subunit comprises an amino acid sequence with at least 85%, or at least 90%, or at least 95%, such as 95%, 96%, 97%, 98%, 99%, or 100% identity to the amino acid sequence of SEQ ID NO: 2.

| | |
|---|---|
| 1 | prwvktvflk llpqvllmrw pldktrgtgs davprglarr |
| | pakgklashg eprhlkecfh |
| 61 | chksnelats krrishqplq wvvensehsp evedvinsvq |
| | fiaenmkshn etkeveddwk |
| 121 | yvamvvdr |

In one embodiment, the α3 subunit of nAChR comprises an amino acid sequence with at least 85%, or at least 90%, or at least 95%, such as 95%, 96%, 97%, 98%, 99%, or 100% identity to the amino acid sequence of SEQ ID NO: 3 (NM_000743.5/NP_000734.2).

| | |
|---|---|
| 1 | mgsgplslpl alspprllll lllsllpvar aseaehrlfe |
| | rlfedyneii rpvanvsdpv |
| 61 | iihfevsmsq lvkvdevnqi metnlwlkqi wndyklkwnp |
| | sdyggaefmr vpaqkiwkpd |
| 121 | ivlynnavgd fqvddktkal lkytgevtwi ppaifkssck |
| | idvtyfpfdy qnctmkfgsw |
| 181 | sydkakidlv ligssmnlkd ywesgewaii kapgykhdik |
| | yncceeiypd ityslyirrl |
| 241 | plfytinlii pcllisfltv lvfylpsdcg ekvtlcisvl |
| | lsltvfllvi tetipstslv |
| 301 | ipligeyllf tmifvtlsiv itvfvlnvhy rtptthtmps |
| | wvktvflnll prvmfmtrpt |
| 361 | snegnaqkpr plygaelsnl ncfsraeskg ckegypcqdg |
| | mcgychhrri kisnfsanlt |
| 421 | rssssesvda vlslsalspe ikeaiqsvky iaenmkaqne |
| | akeiqddwky vamvidrifl |
| 481 | wvftlvcilg taglflqplm areda |

In one embodiment, the 2^{nd} ICL of the α3 subunit comprises an amino acid sequence with at least 85%, or at least 90%, or at least 95%, such as 95%, 96%, 97%, 98%, 99%, or 100% identity to the amino acid sequence of SEQ ID NO: 4.

| | |
|---|---|
| 1 | hyrtptthtm pswvktvfln llprvmfmtr ptsnegnaqk |
| | prplygaels nlncfsraes |
| 61 | kgckegypcq dgmcgychhr rikisnfsan ltrssssesv |
| | davlslsals peikeaiqsv |
| 121 | kyiaenmkaq neakei |

In one embodiment, an exemplary chimeric α6/3 comprises an amino acid sequence with at least 855, or at least 90%, or at least 95%, such as 95%, 96%, 97%, 98%, 99%, or 100% identity to the amino acid sequence of SEQ ID NO: 5.

| | |
|---|---|
| 1 | mltskgqgfl hgglclwlcv ftpffkgcvg cateerlfhk |
| | lfshynqfir pvenvsdpvt |
| 61 | vhfevaitql anvdevnqim etnlwlrhiw ndyklrwdpm |
| | eydgietlrv padkiwkpdi |
| 121 | vlynnavgdf qvegktkall kyngmitwtp paifksscpm |
| | ditffpfdhq ncslkfgswt |
| 181 | ydkaeidlli igskvdmndf wenseweiid asgykhdiky |
| | ncceeiytdi tysfyirrlp |
| 241 | mfytinliip clfisfltvl vfylpsdcge kvtlcisvll |
| | sltvfllvit etipstslvv |
| 301 | plvgeyllft mifvtlsivv tvivlnihyr tptthtmhyr |
| | tptthtmpsw vktvflnllp |
| 361 | rvmfmtrpts negnaqkprp lygaelsnln cfsraeskgc |
| | kegypcqdgm cgychhrrik |
| 421 | isnfsanltr ssssesvdav lslsalspei keaiqsvkyi |
| | aenmkaqnea keivflwvfi |
| 481 | ivcvfgtagl flqpllgntg ks |

In one embodiment, the 32 subunit of nAChR comprises an amino acid sequence with at least 85%, or at least 90%, or at least 95%, such as 95%, 96%, 97%, 98%, 99%, or 100% identity to the amino acid sequence of SEQ ID NO: 6 (NM_000748.3/NP_000739.1).

| | |
|---|---|
| 1 | marrcgpval llgfgllrlc sgvwgtdtee rlvehlldps |
| | rynklirpat ngselvtvql |
| 61 | mvslaqlisv hereqimttn vwltqewedy rltwkpeefd |
| | nmkkvrlpsk hiwlpdvvly |
| 121 | nnadgmyevs fysnavvsyd gsifwlppai yksackievk |
| | hfpfdqqnct mkfrswtydr |
| 181 | teidlvlkse vaslddftps gewdivalpg rrnenpddst |
| | yvditydfii rrkplfytin |
| 241 | liipcvlits lailvfylps dcgekmtlci svllaltvfl |
| | lliskivppt sldvplvgky |
| 301 | lmftmvlvtf sivtsvcvln vhhrspttht mapwvkvvfl |
| | eklpallfmq qprhhcarqr |
| 361 | lrlrrrqrer egagalffre apgadsctof vnrasvqgla |
| | gafgaepapv agpgrsgepc |
| 421 | gcglreavdg vrfiadhmrs edddqsvsed wkyvamvidr |
| | lflwifvfvc vfgtigmflq |
| 481 | plfqnytttt flhsdhsaps sk |

In one embodiment, the 33 subunit of nAChR comprises an amino acid sequence with at least 85%, or at least 90%, or at least 95%, such as 95%, 96%, 97%, 98%, 99%, or 100% identity to the amino acid sequence of SEQ ID NO: 7 (NM_000749.5/NP_000740.1).

| | |
|---|---|
| 1 | mlpdfmlvli vlgipssatt gfnsiaened allrhlfqgy |
| | qkwvrpvlhs ndtikvyfgl |
| 61 | kisqlvdvde knqlmttnvw lkqewtdhkl rwnpddyggi |
| | hsikvpsesl wlpdivlfen |
| 121 | adgrfegslm tkvivksngt vvwtppasyk ssctmdvtff |
| | pfdrqncsmk fgswtydgtm |
| 181 | vdlilinenv drkdffdnge weilnakgmk gnrrdgvysy |
| | pfitysfvlr rlplfytlfl |
| 241 | iipclglsfl tvlvfylpsd egeklslsts vlvsltvfll |
| | vieeiipsss kvipligeyl |
| 301 | lfimifvtls iivtvfvinv hhrssstyhp mapwvkrlfl |
| | qklpkllcmk dhvdrysspe |
| 361 | keesqpvvkg kvlekkkqkq lsdgekvlva flekaadsir |
| | yisrhvkkeh flsqvvqdwk |
| 421 | fvaqvldrif lwlflivsvt gsvliftpal kmwlhsyh |

In one embodiment, the BARP comprises an amino acid sequence with at least 85%, or at least 90%, or at least 95%, such as 95%, 96%, 97%, 98%, 99%, or 100% identity to the amino acid sequence of SEQ ID NO: 8 (XM_017026555.1/XP_016882044.1). Or, the BARP comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 8 and has protein chaperone property, which includes the property of enhancing the expression of the α6 subunit (including chimeric α6/3 subunit as described above) of nAChR.

| | |
|---|---|
| 1 | mgsaqlcagv cdrchtgega geawrvlhsa rgdlrgeagg |
| | apvsrdpglc slkgncgrga |
| 61 | lavppqnphg psnvrgesvp prtppppaha ahlglqsref |
| | qdtpscvpgt sgpgegpppq |
| 121 | frmqptatma taattttttt atvalttswd natgrptaep |
| | dpildnyvll vvvmslfvgg |
| 181 | tlvvlsgvll lckrcwdvhq rlnrameeae kttttyldng |
| | thpaqdpdfr gedpecqdae |
| 241 | terflstsst grrvsfneaa lfeqsrktqd kgrrytlteg |
| | dfhhlknarl thlhlpplki |
| 301 | vtihecdsge assattphpa tspkatlaif qppgkaltgr |
| | svgpssalpg dpynsaagat |
| 361 | dfaeispsas sdsgegtsld agtrstkagg pgaaagpgea |
| | gpgsgagtvl qfltrlrrha |
| 421 | sldgaspyfk vkkwklepsq raasldtrgs pkrhhfqrqr |
| | aasesteqee gdapqedfiq |
| 481 | yiaragdava fphprpflas pppalgrlea aeaaggaspd |
| | sppergagsa gpeqqqpple |
| 541 | pdaerdagpe qaqtsyrdlw slraslelha aasdhsssgn |
| | drdsvrsgds sgsgsggaap |
| 601 | afpppsppap rpkdgearrl lqmdsgyasi egrgagddte |
| | ppaaparprs prawprrprr |
| 661 | dysidektda lfheflrhdp hfddtpaaar hrarahphar |
| | kqwqrgrqhs dpgaraapal |
| 721 | agtpappaga arparaplrr gdsvdgppdg rtlggagddp |
| | aipvieeepg gggcpgsglc |
| 781 | vlpsgsvldk laaglderlf pprlaepvva tpalvaaapt |
| | spdhspa |

In one embodiment, the SULT2β1 comprises an amino acid sequence with at least 85%, or at least 90%, or at least 95%, such as 95%, 96%, 97%, 98%, 99%, or 100% identity to the amino acid sequence of SEQ ID NO: 9 (NM_177973.2/NP_814444.1). Or, the SULT2β1 comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 9 and has protein chaperone property, which includes the property of enhancing the expression of the α6 subunit (including chimeric α6/3 subunit as described above) of nAChR.

| | |
|---|---|
| 1 | mdgpaepqip glwdtyeddi seisqklpge yfrykgvpfp |
| | vglyslesis laentqdvrd |
| 61 | ddifiitypk sgttwmieii clilkegdps wirsvpiwer |
| | apwcetivga fslpdqyspr |
| 121 | lmsshlpiqi ftkaffsska kviymgrnpr dvvvslyhys |
| | kiagqlkdpg tpdqflrdfl |
| 181 | kgevqfgswf dhikgwlrmk gkdnflfity eelqqdlqgs |
| | vericgflgr plgkealgsv |
| 241 | vahstfsamk antmsnytll ppslldhrrg aflrkgvcgd |
| | wknhftvaqs eafdrayrkq |
| 301 | mrgmptfpwd edpeedgspd pepspepepk pslepntsle |
| | reprpnssps pspgqasetp |
| 361 | hprps |

In one embodiment, the LAMP5 comprises an amino acid sequence with at least 85%, or at least 90%, or at least 95%, such as 95%, 96%, 97%, 98%, 99%, or 100% identity to the amino acid sequence of SEQ ID NO: 10 (NM_012261.4/NP_036393.1). Or, the LAMP5 comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 10 and has protein chaperone property, which includes the property of enhancing the expression of the α6 subunit (including chimeric α6/3 subunit as described above) of nAChR.

| | |
|---|---|
| 1 | mdlqgrgvps idrlrvllml fhtmaqimae qevenlsgls |
| | tnpekdifvv rengttclma |
| 61 | efaakfivpy dvwasnyvdl iteqadialt rgaevkgrcg |
| | hsqselqvfw vdrayalkml |
| 121 | fvkeshnmsk gpeatwrlsk vqfvydssek thfkdavsag |
| | khtanshhls alvtpagksy |
| 181 | ecqaqqtisl assdpqktvt milsavhiqp fdiisdfvfs |
| | eehkcpvder eqleetlpli |
| 241 | lglilglvim vtlaiyhvhh kmtanqvqip rdrsqykhmg |

In one embodiment, the CHAT comprises an amino acid sequence with at least 85%, or at least 90%, or at least 95%, such as 95%, 96%, 97%, 98%, 99%, or 100% identity to the amino acid sequence of SEQ ID NO: 11 (NM_020984.4/NP_066264.4). Or, the CHAT comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 11 and has protein chaperone property, which includes the property of enhancing the expression of the α6 subunit (including chimeric α6/3 subunit as described above) of nAChR.

| | |
|---|---|
| 1 | maaktpssee sglpklpvpp lqqtlatylq cmrhlvseeq |
| | frksqaivqq fgapgglget |
| 61 | lqqkllerqe ktanwvseyw lndmylnnrl alpvnsspav |
| | ifarqhfpgt ddqlrfaasl |
| 121 | isgvlsykal ldshsiptdc akgqlsgqpl cmkqyyglfs |
| | syrlpghtqd tlvaqnssim |
| 181 | pepehvivac cnqffvldvv infrrlsegd lftqlrkivk |
| | masnederlp piglltsdgr |
| 241 | sewaeartvl vkdstnrdsl dmierciclv cldapggvel |
| | sdthralqll hgggysknga |
| 301 | nrwydkslqf vvgrdgtcgv vcehspfdgi vlvqctehll |
| | khvtqssrkl iradsvselp |
| 361 | aprrlrwkcs peiqghlass aeklqrivkn ldfivykfdn |
| | ygktfikkqk cspdafiqva |
| 421 | lqlafyrlhr rlvptyesas irrfqegrvd nirsatpeal |
| | afvravtdhk aavpasekll |
| 481 | llkdairaqt aytvmaitgm aidnhllalr elaramckel |
| | pemfmdetyl msnrfvlsts |
| 541 | qvptttemfc cygpvvpngy gacynpqpet ilfcissfhs |
| | cketssskfa kaveeslidm |
| 601 | rdlcsllppt eskplatkek atrpsqghqp |

In one embodiment, the NACHO comprises an amino acid sequence with at least 85%, or at least 90%, or at least 95%, such as 95%, 96%, 97%, 98%, 99%, or 100% identity to the amino acid sequence of SEQ ID NO: 12 (NM_021637.3/NP_067650.1). Or, the NACHO comprises an amino acid sequence with at least 60%, at least 70%, at least 80%, at least 85%, or at least 90%, or at least 95%, such as 95%, 96%, 97%, 98%, 99%, or 100% identity to the amino acid sequence of SEQ ID NO: 12 and has protein chaperone property, which includes the property of enhancing the expression of the α6 subunit (including chimeric α6/3 subunit as described above) of nAChR.

| | |
|---|---|
| 1 | masprtvtiv alsvalglff vfmgtikltp rlskdaysem |
| | krayksyvra lpllkkmgin |
| 61 | sillrksiga levacgivmt lvpgrpkdva nffllllvla |
| | vlffhqlvgd plkryahalv |
| 121 | fgilltcrll iarkpedrss ekkplpgnae eqpslyekap |
| | qgkvkvs |

Any suitable means for introducing heterologous nucleic acid into a cell can be used herein to prepare the recombinant cells disclosed herein, such as DNA transfection (e.g., via a DNA vector) and RNA transduction. In one embodiment, the heterologous nucleic acid to be introduces into cells to generate the recombinant cells are prepared by using a vector, preferably an expression vector. The term “vector” refers to a nucleic acid molecule capable of transporting another nucleic acid to which it has been linked. One type of vector is a “plasmid”, which refers to a circular double-stranded DNA loop into which additional DNA segments can be inserted. Another type of vector is a viral vector wherein additional DNA segments can be inserted. Expression vectors are those vectors capable of directing the expression of genes to which they are operably linked. The expression vectors used herein comprise a nucleic acid encoding a protein sequence in a form suitable for expression of the nucleic acid in a host cell. Thus, the expression vectors can include one or more regulatory sequences, such as a promoter, selected on the basis of the host cells to be used for expression, operably linked to the nucleic acid sequence to be expressed. When used in reference to a expression vector, “operably linked” is intended to mean that the nucleotide sequence of interest is linked to the regulatory sequence(s) in a manner allowing for expression of the nucleotide sequence (e.g., in an in vitro transcription/translation system or in a host cell when the vector is introduced into the host cell). It will be appreciated by those of ordinary skill in the art that the design of the expression vector can depend on such factors as the choice of the host cell to be transformed and the level of expression of protein desired as well as the intended use of the vector.

Any vector known to those skilled in the art in view of the present disclosure can be used, such as a plasmid, a cosmid, a phage vector, or a viral vector. In one embodiment, the vector is an expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein. Conventional cloning techniques or artificial gene synthesis can be used to generate an expression vector according to embodiments of the invention.

Any cell known to those skilled in the art in view of the present disclosure can be used for recombinant expression of α6 (including chimeric α6/3), β2, β3 subunits of nAChR, BARP, SULT2β1, LAMP5, CHAT, and NACHO. In one embodiment, the recombinant cell is a mammalian cell. Suitable the mammalian cells may be selected from human embryonic kidney 293T (HEK293T) cell, HEK293F cells, Hela cells, Chinese hamster ovary (CHO) cells, NIH 3T3 cells, MCF-7 cells, Hep G2 cells, baby hamster kidney (BHK) cells, and Cos7 cells.

Methods of Identifying Agonists, Antagonists, or Positive Allosteric Modulators of α6β2β3 nAChR

Further provided herein are methods of identifying agonists, antagonists, or positive allosteric modulators (PAMs) of an α6-containing nAChR (such as α6β2ß3 nAChR, including chimeric α6/3β2β3 nAChR as described above). PAMs are compounds that bind at sites on the protein surface other than the active sites, and therefore change the conformation of the protein binding sites.

In one embodiment, the method comprises culturing the isolated recombinant cells disclosed herein under conditions where the recombinant cells grow, contacting the recombinant cells with an agent, and determining if the agent is an agonist, antagonist, or PAM of the α6β2β3 nAChR (including chimeric α6/3β2β3 nAChR as described above), wherein an agonist or PAM enhances the activity of the α6β2β3 nAChR (including chimeric α6/3β2β3 nAChR as described above) and an antagonist decreases the activity of the 6β2βnAChR as compared to the activity of the α6β2β3 nAChR (including chimeric α6/3β2β3 nAChR as described above) in a recombinant cell that was not contacted with an agent. Agonists, as used herein, refer to molecules/compounds/peptides that serve to enhance the function of the α6β3β3 nAChR (including chimeric α6/3β2β3 nAChR as described above). PAMs, as used herein, refer to molecules/compounds/peptides that enhance the effect of α6β2β3 nAChR's (including chimeric α6/3β2β3 nAChR as described above) response to a ligand without directly activating the receptor. As used herein, the term “enhance”, “enhanced”, “increase”, or “increased”, when used with respect to α6β2β3 nAChR (including chimeric α6/3β2β3 nAChR as described above) activity refers to an increase in the signaling through the receptor, relative to the corresponding signaling observed in a cell in which an agonist or PAM is not administered. Antagonists, as used herein, refer to molecules/compounds/peptides that serve to block, decrease, or dampen the function of the α6β2β3 nAChR (including chimeric α6/3β2β3 nAChR as described above). In particular embodiments, the agent is a small molecule or peptide.

In one embodiment, FLIPR assay is used to identify agonists of 6β2β3 nAChR (including chimeric α6/3β2β3 nAChR as described above) mediated calcium flux. In this assay, the recombinant cells are incubated with a calcium sensitive dye (such as Ca5), exposed to a test compound, and calcium flux is imaged by FLIPR^{TETRA}.

In one embodiment, FLIPR assay is used to identify antagonists of nicotine-evoked α6β2β3 nAChR (including chimeric α6/3β2β3 nAChR as described above) mediated calcium flux. In this assay, the recombinant cells are incubated with a calcium sensitive dye (such as Ca5). Using a double addition protocol, the recombinant cells are exposed to test compounds during the first duration, and to nicotine (e.g., at EC₈₀ (236 nM)) during the second duration. Thereafter, the calcium flux is imaged by FLIPR^{TETRA}.

In one embodiment, FLIPR assay is used to identify compounds that positively modulate or potentiate nicotine-evoked α6β2β3 nAChR (including chimeric α6/3β2β3 nAChR as described above) mediated calcium flux. In this assay, the recombinant cells are incubated with a calcium sensitive dye (such as Ca5). Using a double addition protocol, the recombinant cells are exposed to test compounds during the first duration, and to nicotine (e.g., at EC₈₀ (236 nM)) during the second duration. Thereafter, the calcium flux is imaged by FLIPR^{TETRA}. If a test compound enhances nicotine-evoked α6β2β3 nAChR (including chimeric α6/3β2β3 nAChR as described above) mediated calcium flux, yet does not enhances calcium flux as an agonist (determined as above), the test compound is termed a positive allosteric modulator (PAM) or potentiator of α6β233 nAChR (including chimeric α6/3β2β3 nAChR as described above).

In a preferred embodiment, the cells are incubated at about 25-35° C. for about 20-50 hr prior to the FLIPR assay or other assay for measuring α6β2β3 nAChR (including chimeric α6/3β2β3 nAChR as described above) activity. In some embodiments, the cells are incubated at about 25° C., about 26° C., about 27° C., about 28° C., about 29° C., about 30° C., about 31° C., about 32° C., about 33° C., about 34° C., or about 35° C., for about 20 hr, about 25 hr, about 30 hr, about 35 hr, about 40 hr, about 45 hr, or about 50 hr prior to the assay.

### Expression System and Kits

Further provided herein are expression systems and kits comprising the isolated recombinant cells. The expression systems and kits may further include instructions for use.

### Example

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Material

| Name | Company | Cat. No |
|---|---|---|
| FreeStyle 293 Expression Media | Gibco | 12338018 |
| FectoPRO Reagent | PolyPlus | 116-010 |
| DMEM-high glucose | Sigma | D5671-500ML |
| Defined FBS | HyClone | SH30070.03 |
| Sodium Pyruvate | HyClone | SH30239.01 |
| DMSO | Sigma Aldrich | 41648 |
| Ca5 dye | Molecular | R8186 |
| | Devices | |
| Nicotine | Tocris | 3546 |
| DHBe | Tocris | 2349 |
| 384-well pp plates | Greiner | 781280 |
| 384-well poly-D-lysine plates | Corning | 354663 |
| Custom HBSS | HyClone | SH3A4475.01 |
| Calcium Chloride | Amresco | E506-500ML |
| Magnesium Chloride | Amresco | E525-500ML |

### Cell Line.

FreeStyle 293F cell line (ThermoFisher Scientific Cat#R79007) Vector Constructs
pcDNA3.1-α6/3 (α6/3 chimera, α6: 1-338; 465-505, α3: 339-464)
pCMV6-XL5-32
pcDNA3.1-3
pcDNA3.1-BARP
pcDNA3.1-NACHO
pcDNA3.1-SULT2β1
pcDNA3.1-LAMP5
pcDNA3.1-CHAT

### Culturing Media

FreeStyle 293 Expression Medium

### Transfection Reagents

FectoPRO DNA Transfection Reagent

### Seeding Media

DMEM+L-glutamine+Sodium Pyruvate
10% FBS
1× Pen/Strep

### Compound Dilution/Ca5 Buffer

HEPES-buffered saline solution (500 ml) supplemented with 1 mM Mg²⁺ and 2 mM Ca²⁺

### Calcium 5 (Ca5) Dye (Molecular Devices)

Calcium dye diluted at 25× concentration in Hanks Buffered Salt Solution. Dye diluted to 1× in HEPES assay buffer with 2 mM CaCl₂) and 1 mM MgCl₂.

### Methods and Procedures

### Day 1 Cell Preparation

Prepare a cell suspension of 1×10⁶ cells per mL by centrifuging cells and resuspending in fresh, prewarmed media. Cell density doesn't need to be readjusted on day of transfection.

### Day 2 Transfection.

The following protocol uses a 100 mL transfection as an example. With 90 mL of cells shaking, prepare the following transfection mix in 10 mL of Freestyle 293 Expression Medium.
Vortex FectoPRO® reagent for 5 sec before adding 75 μl of FectoPRO (0.75 μl reagent per mL of total culture medium) to an empty 50 ml tube.
In a second 50 ml tube, dilute 50 μg of DNA (0.5 μg total DNA per mL of total culture medium; all plasmids 1:1) in Freestyle 293 Expression Medium to a final volume of 10 ml. Vortex gently.
Transfer the diluted DNA to the pure FectoPRO® reagent all at once. Homogenize the solution immediately and incubate for 10 minutes at room temperature.
Transfer the 10 ml FectoPRO®/DNA transfection mix to the cells, homogenize the culture.

### Day 3 Plate Cells

Spin down cells at 300×G for 5 min, aspirate supernatant, and resuspend in 10 ml plating medium.
Seed 25,000 cells/well in 50 μL (5×10⁵cells/mL).
Incubate plates for 24 hours at 30° C. with 5% CO₂ in a humidified atmosphere. Day 4 FLPR Assay
Wash plate with plate washer (4 washes x 100 μL/wash) using compound dilution buffer, leaving 25 μl in each well.
Add 25 μl of 2× Ca5 dye to each well.
Incubate at room temperature for 1 hr.
Wash plate with plate washer (4 washes x 100 μL/wash) using compound dilution buffer, leaving 25 μL in each well.

### Antagonist

Transfer plates to FLIPR for compound addition. 1^{st} addition plate-2× antagonist (25 μL test compound). 2^{nd} addition plate-1× antagonist, 3× agonist (25 μL nicotine EC₈₀) EC₈₀ nicotine 236 nM (25 μL; 3×=708 nM). Control wells for antagonists were in column 23 and 24. Positive control wells received IC100 DHBe (25 μL; 3×=30 UM) in the first addition and negative controls received buffer (25 L). 2^{nd} addition for control wells were IC100 DHBe (10 μM) and EC₈₀ nicotine 236 nM (25 μL; 3×=708 nM).

### Agonist

Transfer plates to FLIPR for compound addition. Single addition plate of 2× agonist (25 μL). Control wells for agonists will be in column 23 and 24. Positive control wells received EC₁₀₀ nicotine (25 μL; 2×=2 μM). Negative control wells received buffer+vehicle (25 μL).
   Functional Expression of α6β2β3 nAChR

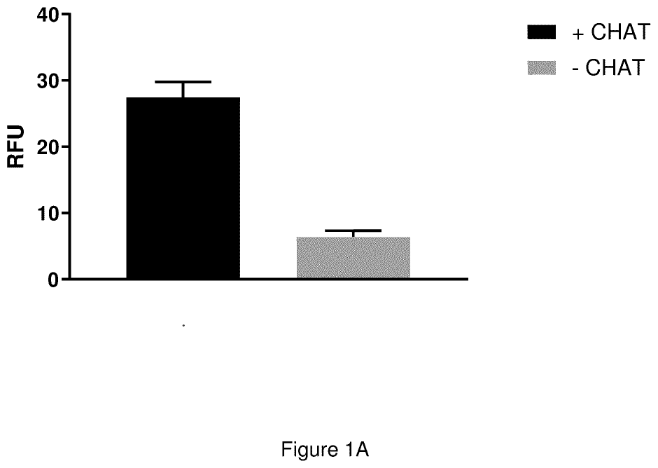

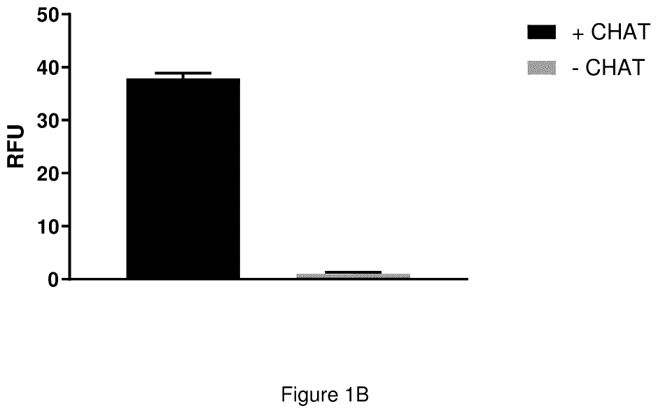

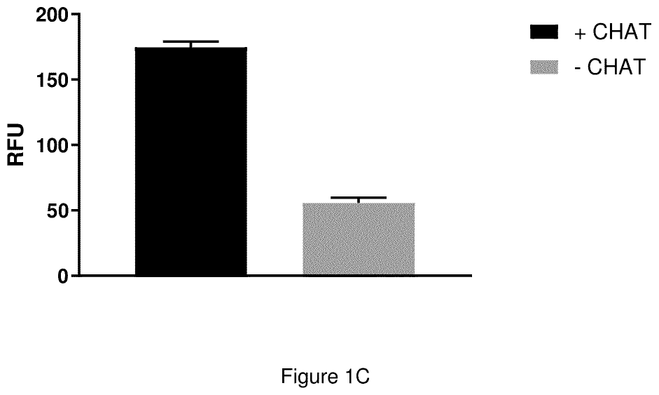

Nucleic acids encoding human α6, 32, and 33 subunits of nAChR were co-transfected with specified combinations of cDNAs (BARP+SULT2β1+LAMP5+NACHO or BARP+SULT2β1+LAMP5+NACHO+CHAT) in HEK293T cells and incubated at 37° C. overnight followed by 30° C. for 24-48 hours. The transfected cells were incubated for one hour at room temperature with Ca5 dye followed by stimulation with Eₘₐₓ nicotine (10 μM). Nicotine-evoked Ca2⁺signal of the transfected are graphed in FIG. 1A. As shown, co-transfection with BARP, SULT2β1, LAMP5, NACHO, and CHAT (marked as “+CHAT”) enhances α6ß233 nAChR function (i.e., nicotine-evoked calcium flux), as compared to co-transfection with BARP, SULT2β1, LAMP5, and NACHO (marked as “-CHAT”).

Functional Expression of α6/3β233 nAChR

Nucleic acids encoding chimeric α6/3 and human 32 and β3 subunits of nAChR were co-transfected with specified combinations of cDNAs (BARP+SULT2β1+LAMP5+NACHO or BARP+SULT2β1+LAMP5+NACHO+CHAT) in HEK293T cells and incubated at 37° C. overnight. The transfected cells were incubated for one hour at room temperature with Ca5 dye followed by stimulation with Eₘₐₓ nicotine (10 μM). Nicotine-evoked Ca2⁺signal of the transfected are graphed in FIG. 1B. As shown, co-transfection with BARP, SULT2β1, LAMP5, NACHO, and CHAT (marked as “+CHAT”) enhances α6/3β2β3 nAChR function (i.e., nicotine-evoked calcium flux), as compared to co-transfection with BARP, SULT2β1, LAMP5, and NACHO (marked as “-CHAT”).

Separately, prior to incubation with Ca5 dye and stimulation with nicotine, the transfected HEK293T cells were incubated at 30° C. for 24-48 hours. Nicotine (10 μM)-evoked Ca²⁺signal of the transfected are graphed in FIG. 1C. Here again, co-transfection with BARP, SULT2β1, LAMP5, NACHO, and CHAT (marked as “+CHAT”) enhances α6/3β2β3 nAChR function (i.e., nicotine-evoked calcium flux), as compared to co-transfection with BARP, SULT2β1, LAMP5, and NACHO (marked as “-CHAT”). In addition, the transfected cells incubated at 30° C. exhibit much higher nAChR function (i.e., nicotine-evoked calcium flux) over those incubated at 37° C., which allow more robust screening for modulators of α6-containing nAChR.

Concentration Response Curve for Antagonist on Nicotine-Evoked Activity of α6/3β2β3 nAChR

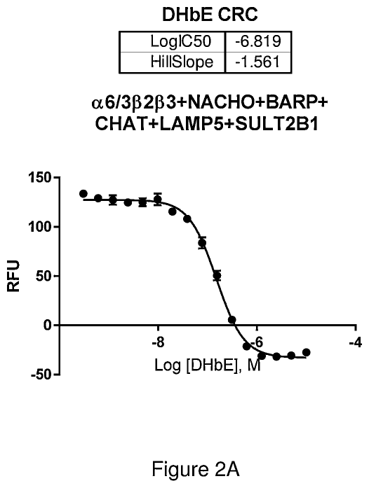

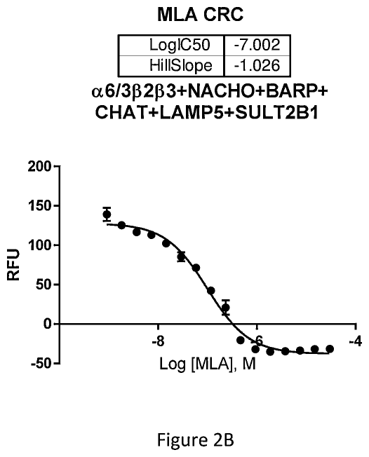

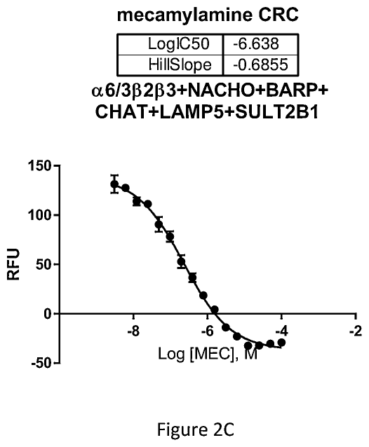

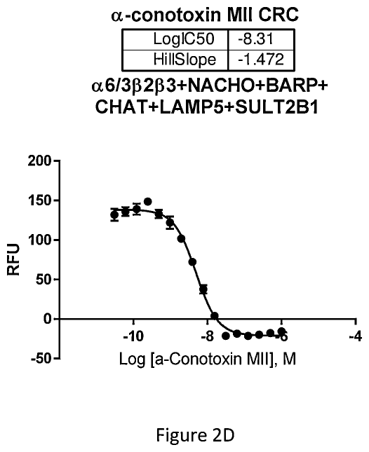

HEK293T cells transfected with nucleic acids encoding α6/3, 2, and β3 subunits of nAChR, along with nucleic acid encoding were BARP, SULT2β1, LAMP5, NACHO, and CHAT, as described above, were incubated at 30° C. for 24-48 hour. The transfected cells were then incubated one hour at room temperature with Ca5 dye followed by stimulation using a two-addition protocol (1^{st} addition: with an antagonist (i.e., DHbE, MLA, mecamylamine, or α-conotoxin MII) at various concentration for 3 min; 2^{nd} addition: with nicotine (EC₈₀, 236 nm) for 3.5 min). Nicotine-evoked calcium flux was then measured using a FLIPR^{TETRA} imager and the final FLIPR signals were averaged and plotted (FIGS. 2A-2D). As shown, each of the tested antagonists decreases the nicotine-evoked calcium flux in a dose-dependent manner.

What is claimed is:

1. An isolated recombinant cell comprising:
a) a heterologous nucleic acid encoding an α6 subunit of nAChR;
b) a heterologous nucleic acid encoding BARP;
c) a heterologous nucleic acid encoding SULT2β1;
d) a heterologous nucleic acid encoding LAMP5;
e) a heterologous nucleic acid encoding CHAT; and
f) a heterologous nucleic acid encoding NACHO.

2. The isolated recombinant cell of claim 1, further comprising:
g) a heterologous nucleic acid encoding a 32 subunit of nAChR; and
h) a heterologous nucleic acid encoding a β3 subunit of nAChR,
wherein, the α6, 32, and β3 subunits of nAChR form an α6β2β3 nAChR.

3. The isolated recombinant cell of claim 1, wherein the α6 subunit of nAChR is an α6/3 chimera in which a full or partial sequence of a second intracellular loop (ICL) of the α6 subunit is replaced by a corresponding sequence of a second ICL of an α3 subunit of nAChR, and wherein the α6/3 chimera and the 2 and β3 subunits of nAChR form a chimeric α6/3β2β3 nAChR.

4. The isolated recombinant cell of claim 1, wherein the recombinant cell is a mammalian cell.

5. The isolated recombinant cell of claim 4, wherein the mammalian cell is selected from the group consisting of a human embryonic kidney 293T (HEK293T) cell, a HEK293F cell, a HeLa cell, a Chinese hamster ovary (CHO) cell, a NIH 3T3 cell, a MCF-7 cell, a Hep G2 cell, a baby hamster kidney (BHK) cell, and a Cos7 cell.

6. The isolated recombinant cell of claim 5, wherein the α6 subunit of nAChR comprises an amino acid sequence with at least 95% identity to the amino acid sequence of SEQ ID NO: 1.

7. The isolated recombinant cell of claim 2, wherein the 32 subunit of nAChR comprises an amino acid sequence with at least 95% identity to the amino acid sequence of SEQ ID NO: 6.

8. The isolated recombinant cell of claim 2, wherein the 33 subunit of nAChR comprises an amino acid sequence with at least 95% identity to the amino acid sequence of SEQ ID NO: 7.

9. The isolated recombinant cell of claim 3, wherein the second ICL of the 6 subunit comprises an amino acid sequence with at least 95% identity to the amino acid sequence of SEQ ID NO: 2 and the second ICL of the α3 subunit comprises an amino acid sequence with at least 95% identity to the amino acid sequence of SEQ ID NO: 4.

10. The isolated recombinant cell of claim 3, wherein the α6/3 chimera comprises an amino acid sequence with at least 95% identity to the amino acid sequence of SEQ ID NO: 5.

11. The isolated recombinant cell of claim 1, wherein the BARP comprises an amino acid sequence with at least 95% identity to the amino acid sequence of SEQ ID NO: 8.

12. The isolated recombinant cell of claim 1, wherein the SULT2β1 comprises an amino acid sequence with at least 95% identity to the amino acid sequence of SEQ ID NO: 9.

13. The isolated recombinant cell of claim 1, wherein the LAMP5 comprises an amino acid sequence with at least 95% identity to the amino acid sequence of SEQ ID NO: 10.

14. The isolated recombinant cell of claim 1, wherein the CHAT comprises an amino acid sequence with at least 95% identity to the amino acid sequence of SEQ ID NO: 11.

15. The isolated recombinant cell of claim 1, wherein the NACHO comprises an amino acid sequence with at least 95% identity to the amino acid sequence of SEQ ID NO: 12.

16. A method for identifying agonists, antagonists, or positive allosteric modulators of α6 containing nAChR, the method comprising:
a) contacting the isolated recombinant cell of claim 1 with an agent; and
b) determining the activity of the α6 containing nAChR of the isolated recombinant cell, wherein the agent is identified as an agonist or positive allosteric modulator (PAM) if the agent enhances the activity of the α6 containing nAChR and the agent is identified as an antagonist if the agent decreases the activity of the α6 containing nAChR as compared to the activity of the α6 containing nAChR when the isolated recombinant cell was not contacted with the agent.

17. A method for identifying agonists, antagonists, or positive allosteric modulators of α6β2β3 nAChR, the method comprising:
a) contacting the isolated recombinant cell of claim 2 with an agent; and
b) determining the activity of the α6β2β3 nAChR of the isolated recombinant cell, wherein the agent is identified as an agonist or positive allosteric modulator (PAM) if the agent enhances the activity of the α6β2β3 nAChR and the agent is identified as an antagonist if the agent decreases the activity of the α6β2β3 nAChR as compared to the activity of the α6β2β3 nAChR when the isolated recombinant cell was not contacted with the agent.

18. The method of claim 17, wherein step b) comprises determining calcium flux of the isolated recombinant cell, wherein the agent is identified as an agonist if the agent enhances the calcium flux as compared to the calcium flux when the isolated recombinant cell was not contacted with the agent.

19. The method of claim 17, wherein step b) comprises determining calcium flux and nicotine-evoked calcium flux of the isolated recombinant cell, wherein the agent is identified as an PAM if the agent does not enhance calcium flux and enhances the nicotine-evoked calcium flux as compared to the calcium flux and nicotine-evoked calcium flux when the isolated recombinant cell was not contacted with the agent.

20. The method of claim 17, wherein step b) comprises determining nicotine-evoked calcium flux of the isolated recombinant cell, wherein the agent is identified as an antagonist if the agent decreases the nicotine-evoked calcium flux as compared to the nicotine-evoked calcium flux when the isolated recombinant cell was not contacted with the agent.
